(19) United States
(12) Patent Application Publication
(10) Pub. No.: US 2011/0047682 A1
(43) Pub. Date: Mar. 3, 2011

(54) Protective Eyewear Device With Lateral Eye Access and Quick Release Mechanism for Interchanging Lenses

(76) Inventor: Arman Hedayat, Windermere, FL (US)

(21) Appl. No.: 12/845,907

(22) Filed: Jul. 29, 2010

Related U.S. Application Data
(63) Continuation-in-part of application No. 12/546,718, filed on Aug. 25, 2009.

(51) Int. Cl. A61F 9/02

(52) U.S. Cl. 2/439; 2/452

(57) ABSTRACT

In one embodiment, the present invention provides an eyewear device for wearing on the face of a user. The device includes a frame body with an interior portion, a ventral side, a dorsal side, a top portion and a bottom portion. The frame body also includes a first lateral appendage, a second lateral appendage, a first top lateral hingepoint and a first bottom lateral hingepoint, a second top lateral hingepoint and a second bottom lateral hingepoint. The frame body additionally includes a first distal side and a second distal side. The frame contacts the face of the user on the dorsal side, except at the first and second distal sides. When the hingepoints of the frame body are engaged, the first and second lateral appendages are rotatable toward said ventral side of the frame body.

### CROSS REFERENCE TO RELATED APPLICATIONS

This application is a continuation-in-part application of U.S. Ser. No. 12/546,718 ('718 app), filed Aug. 25, 2009 to which priority is claimed under 35 USC 120. The disclosure of the '718 app is incorporated herein.

### BACKGROUND OF THE INVENTION

Many different types of eyewear devices are used to protect the eye from foreign matter, while providing a view of the surroundings to the user. Such devices are often used during sports, both for water and non-water sporting activities.

It is often necessary for a user to be able to quickly change the lenses in these types of eyewear devices. Additionally, these eyewear devices often become foggy preventing the user from seeing his surroundings effectively. It is important to prevent fogging of the lenses of eyewear devices as well as provide a means to allow a user to access the eye-area during use of the eyewear device without completely removing the device from use. Restricted access to the eye while the eyewear device is in use is often necessary in order for a user to quickly swipe the eye area when small particulate matter or fluids may encounter the eye during use of the device.

### SUMMARY

In one embodiment, the present invention provides an eyewear device for wearing on the face of a user. The device includes a frame body with an interior portion, a ventral side, a dorsal side, a top portion and a bottom portion. The frame body also includes a first lateral appendage, a second lateral appendage, a first top lateral hingepoint and a first bottom lateral hingepoint, a second top lateral hingepoint and a second bottom lateral hingepoint. The frame body additionally includes a first distal side and a second distal side. The frame contacts the face of the user on the dorsal side, except at the first and second distal sides. When the hingepoints of the frame body are engaged, the first and second lateral appendages are rotatable toward said ventral side of the frame body.

Additionally, the device includes a right lens structure and a left lens structure, both which are slideably held in a groove in the bottom portion of the frame body. The first and second lateral appendages can be rotated about the hingepoints to release the lens structures. A gap is formed between the face of the user and the eyewear device at the first and second distal sides when the eyewear device is in use. The gap allows a user to access the eye in order to remove debris from the eye or wipe the eye while the eyewear device is in use.

In another embodiment, the present invention provides an eyewear lens removal system. The system includes a frame body, made up of a top side, a bottom side, a dorsal side and a ventral side. The frame body also includes a first distal side which contains a first lateral appendage, and a second distal side which contains a second lateral appendage, wherein the first and second lateral appendages associate with the frame body at hingepoints. The system also includes a right lens structure and a left lens structure. The hingepoints of the system may be engaged by rotating the first and second lateral appendages in a ventral direction to release the right and left lens structures. This provides a quick release mechanism, allowing a user to easily remove and replace the lenses in the eyewear device.

In a further embodiment, the invention provides a method for the replacement of lenses on an eyewear device. The method includes the following steps: engaging the hingepoints by rotating the right and left lateral appendages about the hinge in a ventral direction; removing the lenses from the frame body by sliding them away from the center portion of the frame body. The next step involves inserting two new lenses into the groove in the bottom portion of the frame body, and slide the lenses toward the central portion of the frame body, and finally disengaging the hingepoints by rotating the left and right lateral appendages in a dorsal direction to secure the new lenses in place. The left and right lateral appendages may snap when they are rotated back into place once the lenses are secured.

### BRIEF DESCRIPTION OF THE DRAWINGS

A more particular description of the invention briefly described above will be rendered by reference to specific embodiments thereof that are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 depicts a perspective view of an exemplary embodiment of an eyewear device.

FIG. 2 depicts a rear side view of the frame body of an exemplary embodiment of an eyewear device.

FIG. 3 shows a rear side view of an exemplary embodiment of an eyewear device with the hingepoints engaged to allow removal and/or replacement of the lens structures.

FIG. 4 represents a rear view of an exemplary embodiment of the frame body.

FIG. 5 depicts a perspective view of an exemplary embodiment of both right and left lens structures.

FIG. 6 represents a cross-section view of the device shown in FIG. 4 along the plane x-x.

FIG. 7 represents a side perspective view of an embodiment in use.

### DETAILED DESCRIPTION OF THE DRAWINGS

A common problem associated with most eyewear devices, particularly those that are worn close to the face such as sport goggles, is that they tend to fog while in use. There are many different types of anti-fogging mechanisms manufactured for use in goggles in order to counteract this problem. However, they often malfunction, and as with all mechanisms, they require maintenance. Also, the inventor has realized an additional problem with these goggles, particularly when they used in water sports, water tends to accumulate inside the goggle between the user's face and the lens. This obstructs the ability of the user to see clearly, as well as resulting in discomfort for the user.

An additional concern associated with the use of goggles, is that often debris or water gets trapped inside the goggle and cannot be removed without removing the entire goggle from the face of the user. The ability of the user to access the protected eye area if necessary to swipe the water or debris away without removal of the entire goggle is ideal.

A further concern with goggles and other eyewear devices is the inability to easily remove and replace the lenses when necessary. Typically, the removal of lenses from eyewear devices requires certain assembling or disassembling tools such as miniature screw-drivers or other small difficult-to-find contraptions. These type of tools are not always readily available to a user when a lens needs to be exchanged. Removing or replacing lenses in eyewear is often inconvenient and time consuming.

In accordance with one aspect of the present invention, there is provided a device configured to protect the eyes of a user from debris and other foreign bodies, while also providing eye-access to the user while in use. The inventor has recognized a need for such a device, particularly for use in sports such as wakeboarding, kite boarding, and other water-sport activities where a completely sealed goggle structure is not necessary, but eye protection would be advantageous. In such water-sport activities, protection to the eye from both wind and foreign particles is ideal, and quick and easy restricted access to the eye is beneficial. The inventor has also recognized a need for such a device during non-water sport activities such as racquetball or tennis. The device would be useful in order to protect the eye, but also allow a user quick access to the eye area through a restricted opening in order to wipe away any sweat or other particulate matter that may encounter the eye.

In one embodiment, the invention pertains to an eyewear device for wearing on the face of a user, which allows the user to access the eye without removing the device. In a particular embodiment the device includes a frame body with an interior portion, a ventral side, a dorsal side, a top portion and a bottom portion. The frame also includes a first lateral appendage and a second lateral appendage, a first top lateral hingepoint and a first bottom lateral hingepoint, a second top lateral hingepoint and a second bottom lateral hingepoint. The frame also includes a first distal side and a second distal side, and is configured such that the frame contacts the face of the user on said dorsal side, except at the first and second distal sides of the frame body. Moreover, when the hingepoints of the frame are engaged, the first and second lateral appendages are rotatable toward the ventral side. Additionally, the device also includes a right lens structure and a left lens structure, which are slideably held in a groove in the bottom portion of the frame body. The first and second lateral appendages can be rotated about the hingepoints providing release of the lens structures. The device provides a gap, formed between the face of the user and the eyewear device at the first and second distal sides of the frame body when the eyewear device is in use. In an alternative embodiment, the frame body is flexible.

In a more particular embodiment, the hingepoints rotate ventrally at least 20 degrees, and more preferably 30-90 degrees. In yet another embodiment, the right and left lens structures have a distal and a proximal side. The distal side is greater in length than the proximal side of each lens structure. In still another embodiment, the interior portion of the frame body contains a groove pocket at the top portion and the bottom portion of the frame body. The shape and structure of the lens allows it to be easily removed from or inserted into the frame body by simply sliding it one way or the other within the groove pocket in the interior portion of the frame body.

In a more specific embodiment, the gap is from about 1 centimeter to about 5 centimeters, or any 0.2 centimeters in between. In another embodiment of the present invention, a flexible band attaches to the first and second lateral appendages to secure the eyewear device onto the face of the user.

In yet another embodiment, an eyewear lens removal system is provided. The system includes a frame body with a top side, a bottom side, a dorsal side and a ventral side. The frame also contains a first distal side with a first lateral appendage, and a second distal side with a second lateral appendage, the first and second lateral appendages associate with the frame body at hingepoints. The system also includes a right lens structure and a left lens structure. The hingepoints may be engaged by rotating the first and second lateral appendages in a ventral direction in order to release the right and left lens structures for replacement of the lenses. In a more specific embodiment, the right and left lens structures of the system are secured within the frame body in a groove pocket.

In another embodiment, the distance between the distal side of the frame body and the hingepoint is “y”. In a more particular embodiment, the distance “y” is between 3-5 millimeters in length. In an alternative embodiment, “y” extends from the distal side of the frame body to about 2 millimeters inside the distal side of the lens structure.

In another embodiment of the present invention, a method is provided for the replacement of lenses on an eyewear device. The method includes first engaging the hingepoints by rotating the right and left lateral appendages about the hinge in a ventral direction; removing the lenses from the frame body by sliding them away from the center portion of the frame body. The method also includes inserting two new lenses into the groove in the bottom portion of the frame body, and sliding the lenses toward the central portion of the frame body; and finally disengaging the hingepoints by rotating the left and right lateral appendages in a dorsal direction to secure the new lenses in place. Once the lenses are secured in place, the left and right lateral appendages may snap into position.

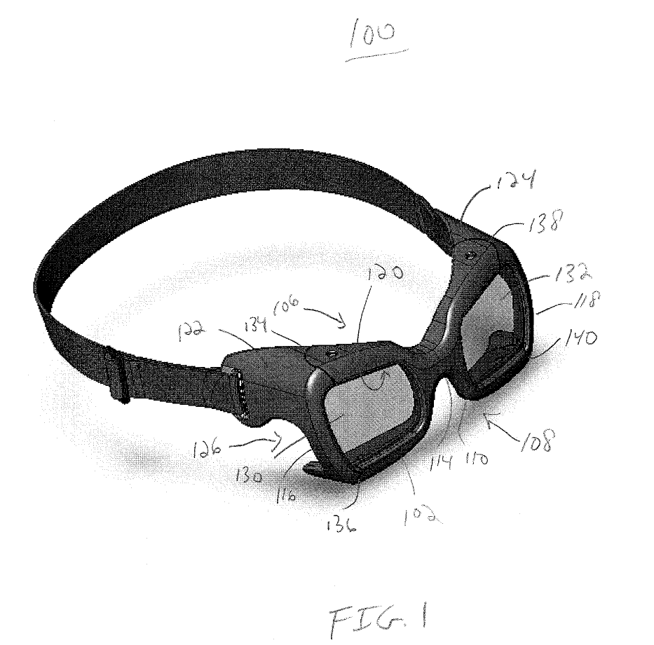

Turning now to the drawings, FIG. 1 shows a perspective view of an exemplary embodiment of an eyewear device 100. The device includes a frame body 102, with a top portion 106, a bottom portion 108, a first distal side 116 and a second distal side 118. The frame body also includes a ventral side 110, and a center portion 114, as well as an interior portion 120. Associated with the frame is a first lateral appendage 122 and a second lateral appendage 124, wherein the intersection between the first lateral appendage 122 and the first distal side of the frame 116 forms a right lateral gap 126, and the second lateral appendage 124 intersects with the second distal side of the frame 118 (behind the device in the picture) to form a left lateral gap.

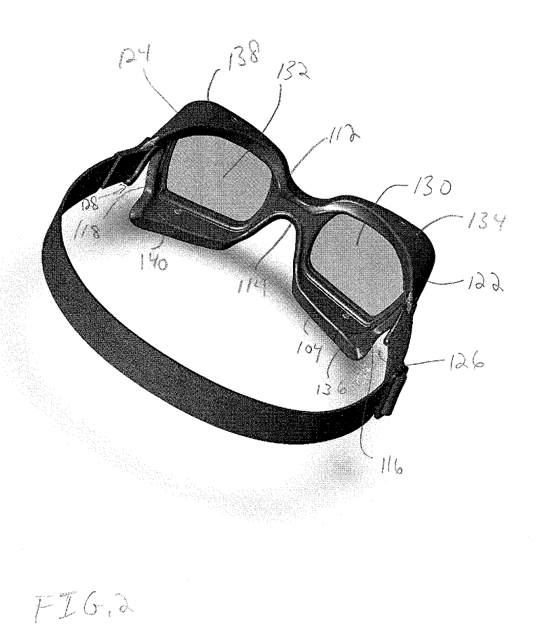

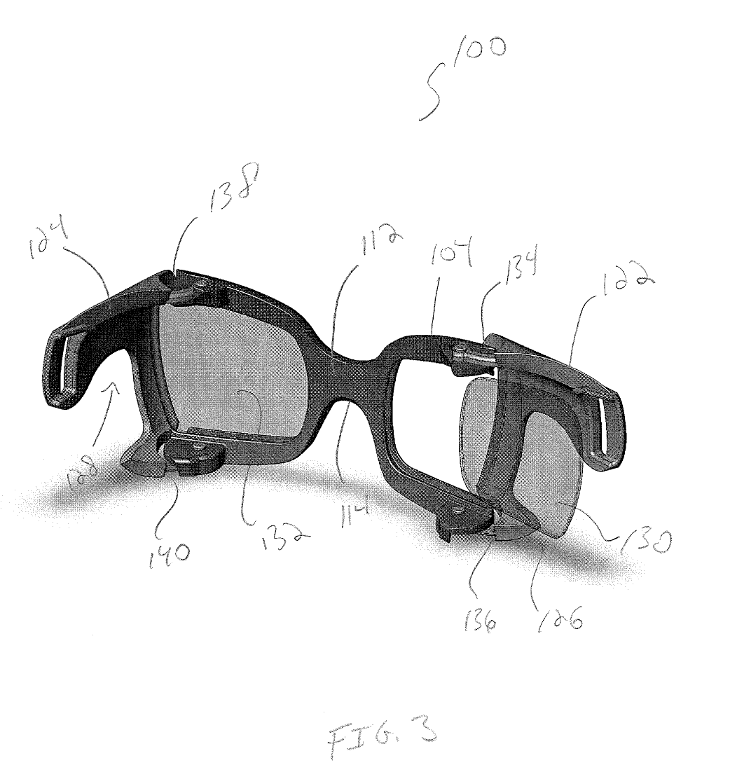

FIG. 2 depicts the dorsal side 112 in a rear side view of the frame body 102 of an exemplary embodiment of an eyewear device 100. The first top lateral hingepoint 134 and first bottom lateral hingepoint 136, as well as the second top lateral hingepoint 138 and the second bottom lateral hingepoint 140 are shown. All hingepoints in this figure are shown as disengaged. A right lens structure 130 and a left lens structure 132 are seen in the figure surrounded by the frame body 102. Both the right lateral gap 126 and the left lateral gap 128 are in view in this figure. In an optional embodiment, a cushion pad layer may be applied to the region 104 layering the dorsal side 112, so as to increase comfort and to provide contact between the face of a user and the frame body 102 of the device 100. FIG. 3 shows the dorsal side 112, a rear side view of an exemplary embodiment of an eyewear device 100, with the hingepoints 134, 136, engaged to allow removal and/or replacement of the lens structure 130.

FIG. 4 represents a rear view of an exemplary embodiment of the frame body 102, showing the acute angles formed by the top portion 106 and the bottom portion 108 of the frame with the first and second distal sides of the frame 116, 118. An x-x axis is also represented in FIG. 4.

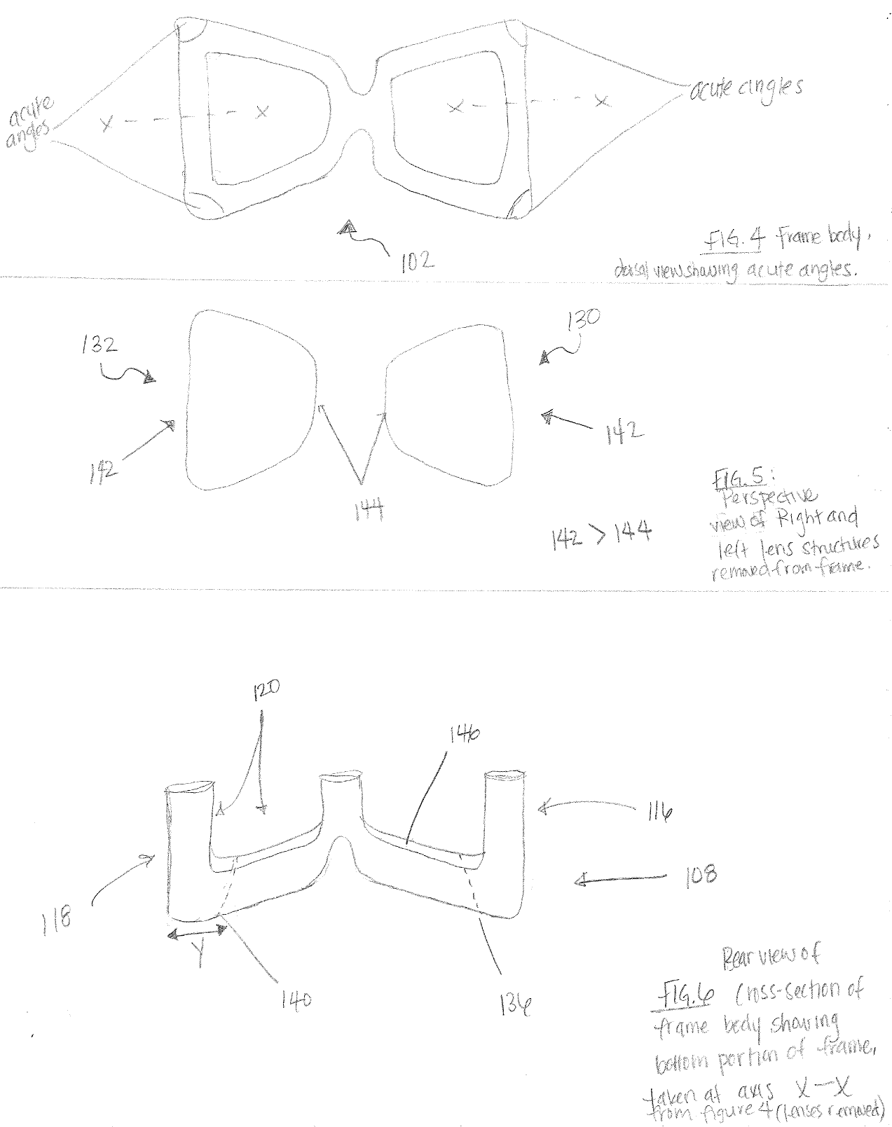

FIG. 5 depicts a perspective view of an exemplary embodiment of both right and left lens structures, 130 and 132. The embodiment represents the distal sides of the lens structures 142 are greater in length than the proximal sides of the lens structures 144 in order to facilitate easier removal of the lenses 130, 132 from the frame body 102.

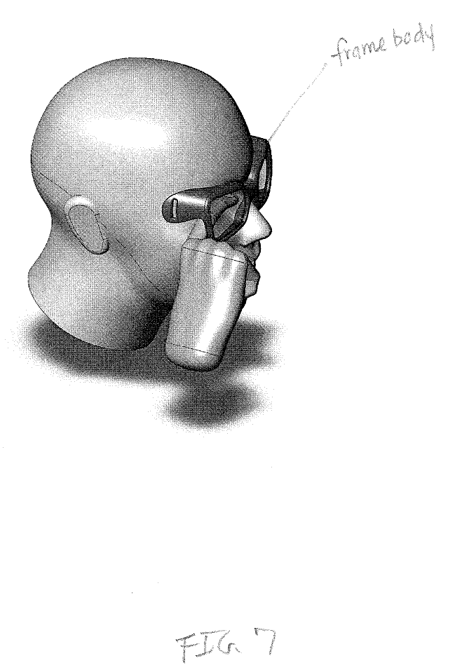

FIG. 6 represents a cross-section view of the device 100 shown in FIG. 4 along the plane x-x, with the groove pocket 146 visible within the interior portion 120 of the frame body 102. The groove pocket 146 assists in keeping the lens structures 130, 132 in place within the frame body 120 of the eyewear device 100.

In another embodiment, the invention pertains to an eyewear device that has the structural shape and configuration of that shown in FIG. 1, but which lacks hingepoints and a lens release mechanism.

FIG. 7 shows a eyewear device shown in a model to illustrate how the shape and configuration of the device facilitate access to the eyes for wiping and cleaning but which also provide protection to the eye.

The disclosures of the cited patent documents, publications and references are incorporated herein in their entirety to the extent not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims

What is claimed is:

1. An eyewear device for wearing on the face of a user, said device comprising:
a frame body comprising an interior portion, a ventral side, a dorsal side, a top portion and a bottom portion, a first lateral appendage and a second lateral appendage, a first top lateral hingepoint and a first bottom lateral hingepoint, a second top lateral hingepoint and a second bottom lateral hingepoint, a first distal side and a second distal side, wherein the frame contacts the face of the user on said dorsal side, except at the first and second distal sides; wherein when said hingepoints are engaged, said first and second lateral appendages are rotatable toward said ventral side;
a right lens structure and a left lens structure slideably held in a groove in said bottom portion of said frame body, whereby said first and second lateral appendages can be rotated about said hingepoints to release said lens structures;
a gap is formed between the face of the user and the eyewear device at said first and second distal sides when said eyewear device is in use.

2. A device as in claim 1, wherein said frame body is flexible.

3. A device as in claim 1, wherein said hingepoints rotate ventrally at least 70 degrees.

4. A device as in claim 1, wherein the top portion and the distal sides of the frame body join at acute angles.

5. A device as in claim 1, wherein the bottom portion and the distal sides of the frame body join at acute angles.

6. A device as in claim 1, wherein the right and left lens structures have a distal and a proximal side, whereby the distal side is greater in length than the proximal side.

7. A device as in claim 1, wherein the interior portion of the frame body contains a groove pocket at the top portion and the bottom portion of the frame body.

8. A device as in claim 1, wherein said gap is from about 1 centimeter to about 5 centimeters, or any 0.2 centimeters in between.

9. A device as in claim 1, wherein a flexible band attaches to the first and second lateral appendages to secure the eyewear device onto the face of the user.

10. An eyewear lens removal system, comprising:
a frame body, comprising a top side, a bottom side, a dorsal side and a ventral side, a first distal side containing a first lateral appendage, and a second distal side containing a second lateral appendage, wherein said first and second lateral appendages associate with said frame body at hingepoints;
a right lens structure and a left lens structure;
wherein said hingepoints may be engaged by rotating said first and second lateral appendages in a ventral direction to release said right and left lens structures.

11. A system as in claim 10, wherein the right and left lens structures are secured within the frame body in a groove pocket.

12. A system as in claim 10, wherein the distance between the distal side of the frame body and the hingepoint is “y”.

13. A system as in claim 12, wherein “y” is between 3-5 millimeters in length.

14. A system as in claim 12, wherein “y” extends from said distal side of the frame body to about 2 millimeters inside the distal side of the lens structure.

15. A method for the replacement of lenses on an eyewear device, comprising the steps:
engaging the hingepoints by rotating the right and left lateral appendages about the hinge in a ventral direction;
removing the lenses from the frame body by sliding them away from the center portion of the frame body;
inserting two new lenses into the groove in the bottom portion of the frame body, and sliding the lenses toward the central portion of the frame body;
disengaging the hingepoints by rotating the left and right lateral appendages in a dorsal direction to secure the new lenses in place.
